(19) United States
(12) Patent Application Publication
(10) Pub. No.: US 2015/0320930 A1
(43) Pub. Date: Nov. 12, 2015

(54) RETAINING DEVICE FOR HOLDING ITEMS ON A HOSPITAL BED FRAME

(71) Applicant: Edwin Lynn James, Alamo, CA (US)

(72) Inventor: Edwin Lynn James, Alamo, CA (US)

(21) Appl. No.: 14/802,891

(22) Filed: Jul. 17, 2015

Related U.S. Application Data
(63) Continuation-in-part of application No. PCT/US2014/012093, filed on Jan. 17, 2014.
(60) Provisional application No. 61/754,547, filed on Jan. 19, 2013.

(51) Int. Cl. A61M 5/14; A61B 19/02; A61G 7/05

(52) U.S. Cl. CPC A61M 5/1418; A61G 7/05; A61B 19/0256

(57) ABSTRACT

A device for holding items on a portion of a hospital bed frame comprises an attachment part that may include a mounting base having a channel or groove on an underside for mounting to a portion of a bed frame, or an elongated hollow and open cylinder made of a flexible material designed for clipping to a portion of a hospital bed frame, and further including a holding part connected to the attachment part and designed for holding at least one item. The holding part, in certain aspects, rotates 360° to allow for proper orientation of lines, hoses and tubing for enhanced patient comfort and optimized performance.

### CROSS-REFERENCE TO RELATED APPLICATIONS

This patent application is a continuation-in-part of International Application PCT2014/012093, filed on Jan. 17, 2014, which claims the benefit of U.S. Provisional Patent Application No. 61/754,547, filed Jan. 19, 2013, each of which is incorporated by reference in its entirety for all purposes.

### FIELD

The present application relates to the field of attachment devices and more particularly to devices for holding items on a hospital bed, for example cables, tubes or a remote controller.

### BACKGROUND

Hospital patients are commonly treated with fluids via tubes, such as for instance i.v. tubing for parenteral medication, dripping tubes to supply serum, nasogastric and/or stomach tubes, catheters etc. Hospital patients are also commonly supplied with gas, such as for example oxygen through oxygen tubes while confined on hospital beds. Further, many hospital beds comprise mechanisms for adjusting inclination or height of the beds which require the presence of electric cables or wires around the bed's frame to accommodate a bed remote contoller. A bed remote controller usually also includes a nurse call button. Many hospital rooms are further equipped with televisions and there is a need to put the TV remote controller near the bed, so that it is accessible to a patient lying in bed. As a consequence, a substantial number or items, such as cables, tubes, wires, remotes controllers, etc. can be present around a hospital bed at any time.

Hospital workers, in particular nurses who install these tubes, cables wires and other items, are faced with the problem of finding an adequate and safe way to secure them to a portion of the hospital bed so that the tubes do not fall on the ground or get disrupted by visitors or hospital personnel present around the bed. A loose cable or tube around a hospital bed can represent a hazard. It can represent a hazard for the patient first, as if not properly secured, the tube can be accidentally stripped off the patient, depriving the patient from needed medicine or causing an injury while being pulled off by someone tripping on the tube. A typical situation is a nurse, doctor or visitor accidentally getting caught in a loose tube. The visitor may as a result fall down with a risk of injury while tearing the tube off the patient's body, also causing injury to the patient. To make sure that this does not happen, nurses have to secure such tubes and cables to a portion of the hospital bed. However, nowadays, nurses lack an adequate device to achieve this and one of their current precarious solutions is to tape a tongue depressors or plastic Dixie cups taped to the bed rails in an attempt to lift and route cords, tubes and wires off the floor for safety and to position the tubes and cords nearer to the patient. FIGS. 1 and 2 show examples of prior art systems that nurses are using. FIG. 1 shows a prior art system that nurses use to secure electric and other lines and cables on a portion of a hospital bed by taping down a tongue depressors (P2), with tape (P1) on a portion of a hospital bed (P3) to hold in place cables (P4). Likewise, FIG. 2 shows another prior art system that nurses use to secure cables around a hospital bed. In this system, a plastic Dixie type cup (P7) is taped upside down with tape (P6) on a portion of a hospital bed (P8) to hold cables (P5) in place. These systems, although inadequate, time consuming and unsafe are part of common and normal procedure in hospitals today. The taping of these makeshift type items are dangerous and ineffective in securing necessary items. Also items taped for periods of time can be contaminated and harbor infectious diseases and therefore need disinfecting, cleaning or replacement after each use.

From a patient point of view, it is very important to have easy and quick access to the nurse call button, especially in case of emergency. There is therefore a need for a safe harbor for the nurse call button, which is often located on a remote control. One problem is that said remote control is often found hooked beneath or between the bed's frame and safety rails, making the nurse call button unavailable to patients when it is needed most.

Therefore, there is a need for a safe and positive securement of these hospital items around a hospital bed, to prevent care workers or guests tripping on tubes and cords hanging from hospital bed to floor and provide a secure place for all items around the hospital bed, including for a nurse call button. Without adequate solution to this need, both patients and caregivers are faced with numerous safety issues including those mentioned above.

Devices for holding items on a hospital bed are known, for example published US patent application No. 2006/0031988 discloses a device that comprises an adhesive tab to be fixed on a hospital bedrail. Because it uses an adhesive tab, this device presents a drawback of leaving adhesive material on the hospital bed once the device has been removed. Moreover, if removed and repositioned several times, which is a common situation in hospital setting, the adhesive tab tends to lose its adhesiveness and the device can no longer be properly secured on the hospital bedrail. Further, because it uses adhesive material, this device cannot be properly decontaminated and may induce nosocomial cross-contamination between patients when reused, which is highly undesirable. Also this device cannot rotate to better position devices. Also this device can only drape cords, wires and small tubes, and it cannot hold in position all the small tubes, wires and cords.

There is a need for better devices for patients, visitors and healthcare personnel safety, better caretaker access, better patient comfort and less patient anxiety as well as healthier environment from infectious diseases harbored in sticky tape residue on beds. These devices are also very low cost to health care people and are cost effective and healthier to be disposed after each patient use. There is a need for a better device.

### SUMMARY

In one embodiment, a device is provided for holding an item onto a portion of a hospital bed, such as a bed rail. The device typically includes an attachment element rotatably connected to a holding element such that the holding element is able to rotate with respect to the attachment element. The holding element typically includes a receiving and retention element, such as a foam or rubber material, that holds at least one item such as a tube, an electrical cable, a remote control device, etc. The attachment part, in one embodiment, includes a mounting base element configured to attach to or mate with the contour of a bed frame portion, such as a bed rail. In certain aspects, the mounting base includes a flat element having a channel or groove on an underside portion, wherein the channel or groove on the underside portion facilitates attachment to the bed frame portion. For example, the groove or channel allows for a better fit for a bed frame portion that has a curved, rounded or somewhat rounded contour. A tape material, such as a cohesive tape, secures the attachment portion to the bed frame portion. Use of a cohesive tape provides for robust retention as well as prevents sticky gum residue buildup on the bed frame portion upon removal. In another embodiment, the attachment element includes a clipping element that enables clipping to the portion of the bed frame to secure the device thereto. For example, in certain aspects, the clipping element may include an elongated hollow an open cylinder made of a semi-flexible material. In certain aspects, the holding element may include a clipping element that is adapted to securably retain an item such as a cable or a tube.

In another aspect, a device is provided for holding items onto a portion of a hospital bed comprising a clipping part comprising an elongated hollow and open cylinder made of a flexible material designed for clipping to a portion of a hospital bed, said device also comprising a holding part connected to the clipping part and designed for holding at least one item, said holding part being able to rotate with regard to the clipping part. In certain aspects, the holding part includes a receiving and retention element which may include a foam or rubber material for holding the at least one item.

In another aspect, a kit is provided. The kit typically includes elements of a retention device for holding an item on a portion of a bed frame. The kit typically includes an attachment element adapted to attach to a portion of a hospital bed frame, and a holding element adapted to be rotatably connected to said attachment element. The holding element may form, or it may include, a receiving and retention element for holding at least one item. The holding element is able to rotate with regard to the attachment element when connected thereto. The kit further typically includes an adhesive mechanism such as a tape element adapted for securing the attachment element to the portion of the bed frame.

Another aspect relates to the use of a devices according to the invention.

### BRIEF DESCRIPTION OF THE FIGURES

FIG. 1 shows a drawing of a prior art device taped for holding cables and tubes to a portion of a hospital bed and holding cables and/or tubes.

FIG. 2 shows a drawing of a prior art device taped for holding cables and tubes to a portion of a hospital bed and holding cables and/or tubes.

FIG. 3 shows a perspective view of an embodiment of a device according to the present disclosure.

FIG. 4 shows a perspective view of the embodiment of the device shown in FIG. 3 when in use.

FIG. 5 shows an exploded view of an embodiment of the device shown in FIG. 3 or 4.

FIG. 6 shows a perspective view of an embodiment of an attachment part of a device according to the present disclosure.

FIG. 7 and drawings 7A, 7B, 7C, 7D and 7D shows cross-sectional views of various embodiments of attachment parts of a device according to the present disclosure.

FIG. 8 shows a perspective view of another embodiment of a device according to the present disclosure.

FIG. 9 shows a perspective view of two different embodiments of a device according to the present disclosure when in use.

FIG. 10 shows a perspective view of yet another of a device according to the present disclosure for safe harbor of handset that often contains the nurse call button and TV or radio controls.

FIG. 11 shows an exploded view of the device shown in FIG. 10.

FIG. 12 shows a perspective view of the device shown in FIGS. 10 and 11 when in use.

FIG. 13 shows a perspective view of an embodiment of a device according to the present disclosure.

FIG. 14 shows an embodiment of a retention device including a holding part configured similar to the holding part shown and described above with reference to FIG. 8.

FIG. 15 shows an embodiment of a retention device including a holding part configured similar to the holding part shown and described above with reference to FIG. 10

FIG. 16 A-C illustrate embodiments of retention device secured to a bedrail using a cohesive tape.

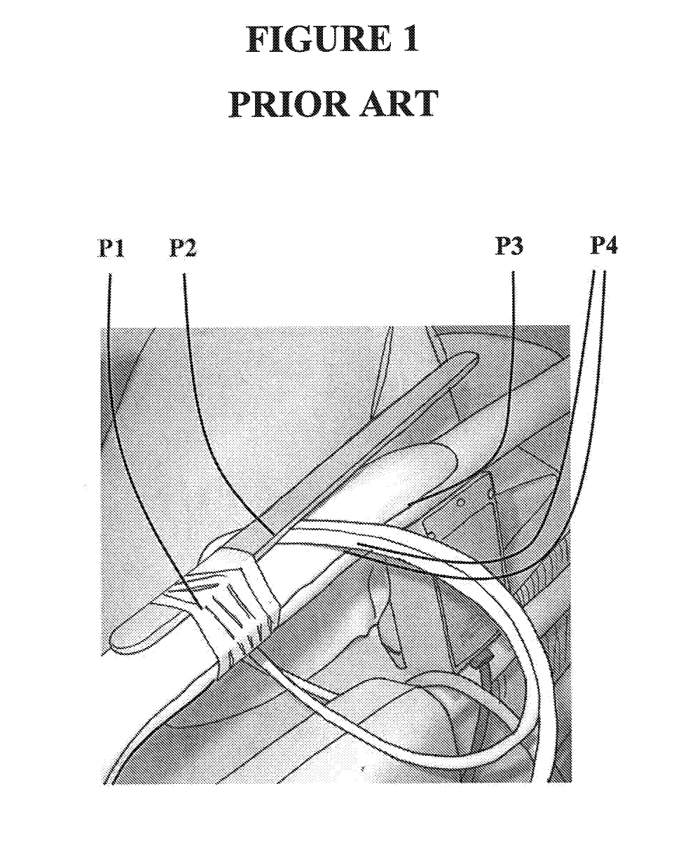

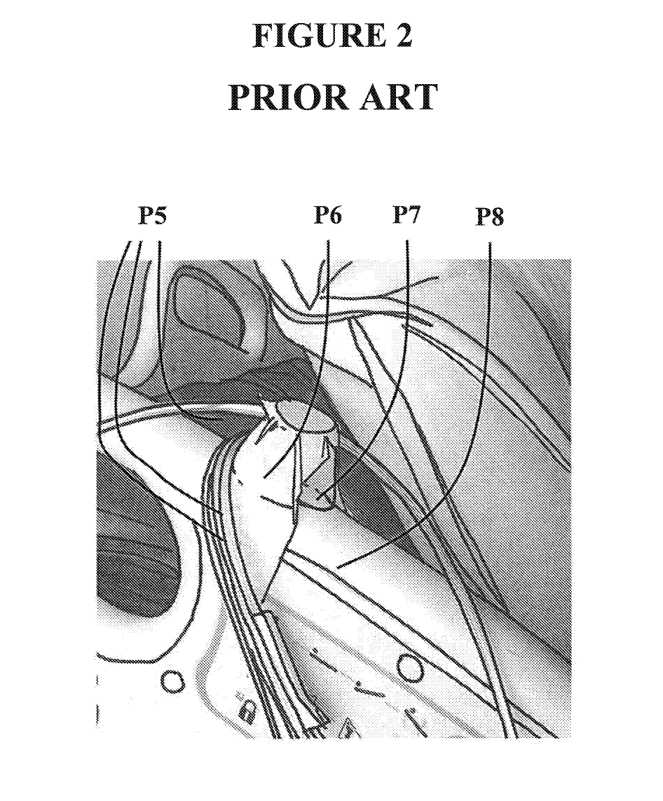

### DETAILED DESCRIPTION

A device for holding items on a portion of a hospital bed frame comprises an attachment part. The attachment part may include a substantially flat mounting base with a mating groove or channel on an underside portion that contacts a portion of the bed frame. The attachment part may include an elongated hollow and open cylinder made of a semi-flexible or flexible material designed for clipping to a portion of a hospital bed frame. The device further includes a holding part connected to the attachment part for holding at least one item. The holding part can, in certain aspects, rotate 360° relative to the attachment part to allow for proper orientation of lines, hoses and tubing for enhanced patient comfort and optimized performance. In certain aspects, the devices of the present disclosure are disposable (e.g., easy to remove and discard and replace with another device).

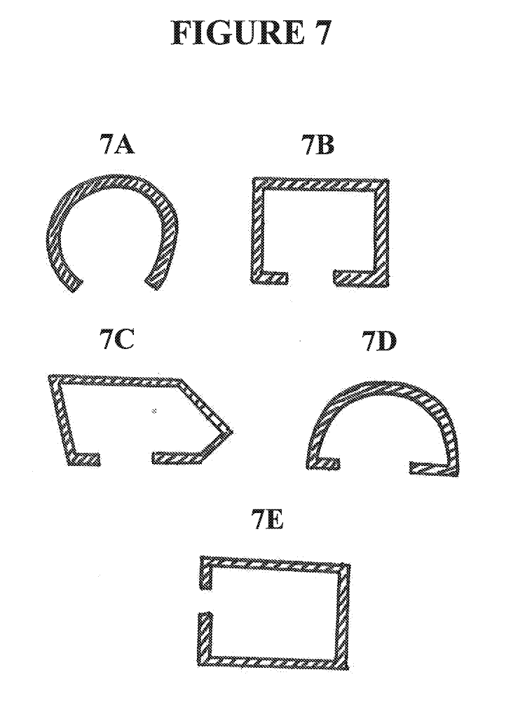

As used herein, the term “cylinder” means the surface generated by an outer straight line intersecting and moving along a plane, while remaining parallel to an inner fixed straight line that is not on or parallel to the plane. It is to be understood that within the scope of the present disclosure, the term cylinder is not limited to right circular cylinders, i.e. cylinders that have a circular cross section in a plane that is perpendicular to the fixed straight line. The term cylinder with the scope of the present disclosure also encompass cylinders having a cross section in a plane perpendicular to said fixed straight line that can be chosen from, but not limited to: a triangle, a square, a rectangle, a parallelogram, a pentagon, an hexagon, a circle, an oval, or an ellipse. Illustrative and non-limiting examples of such cylinders are shown with cross-sectional views in FIG. 7, in particular in drawings 7A to 7E. The cylinders shown in FIG. 7 are open as defined herein.

The term “remote control” or “remote” or “remote controller” as used herein denotes wireless as well as hardwired remote control units. Such remote control units can include a nurse call button.

The term “open cylinder” as used herein means a cylinder as defined herein that comprises an opening in its surface, e.g., an opening along a portion of the cylinder parallel to the cylindrical axis.

The term “at least one” or “one or more” means one or more, for example and without limitation, 1, 2, 3, 4, 5, 6, 7, 8, 9 or 10 or more elements.

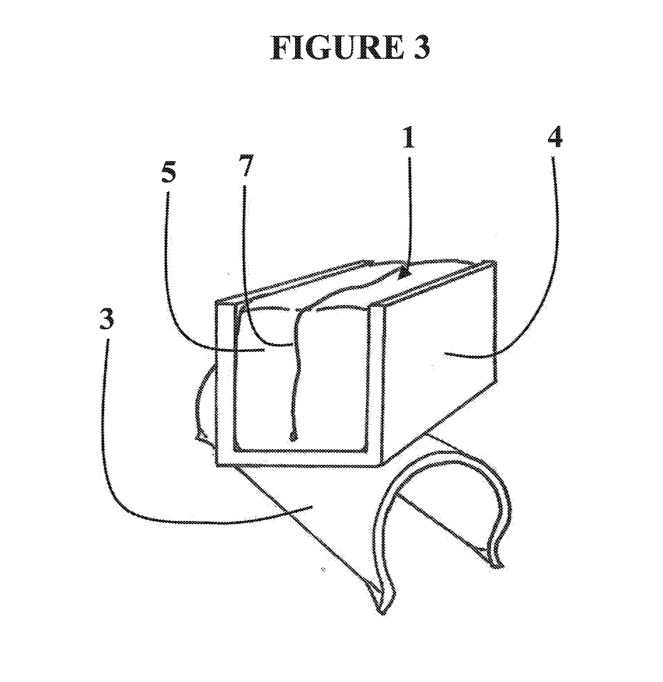

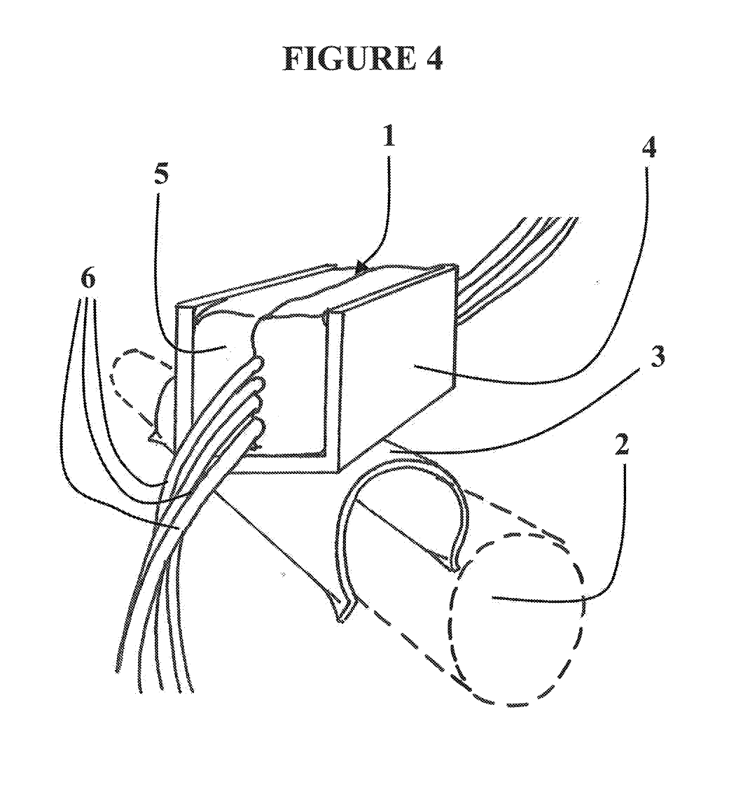

Referring to FIG. 3, an embodiment of a device (1) for holding items is shown. The device includes an attachment element such as clipping part (3) that comprises an elongated hollow and open cylinder made of a flexible or semi-flexible material designed for clipping to said portion (2) of a hospital bed frame (shown in FIG. 4). The material can be flexible with memory, such as polyvinyl chloride (PVC) or other plastic material that has similar physical properties. Extruded or molded PVC pieces can be used. The clipping part (3) can, for example, be made from a piece of extruded PVC tube by cutting or otherwise forming an opening in the wall so as to obtain an open cylinder. In certain aspects, the clipping part (3) includes a portion (12) that is slightly bent toward the outside (or flared outward relative to its center length axis) at the edges of the opening so as to facilitate clipping to a receiving element such as a bed rail. In other aspects, clipping part (3) includes a portion (12) that has rounded edges to facilitate clipping to a receiving element, for example as shown in FIG. 7.

The device (1) further includes a holding part (4) connected to the clipping part (3) and designed for holding at least one item (not shown on FIG. 3). Connection of the clipping part (3) with the holding part (4) can be done in any of a variety of ways. In an embodiment, the holding part (4) is designed to be able to rotate with regard to the clipping part (3). This can, for example, be achieved by connecting the holding part (4) and the clipping part (3) with a connection element or connecting means that allow for a rotation of the holding part relative to the clipping part, for example and without limitation, a rivet, nut and bolt, etc.

In the embodiment shown on FIG. 3, the holding part (4) is substantially U-shaped (cross-sectional shape) with two parallel side walls connected to a bottom wall (bottom refers to the direction of the device when in typical use). The holding part can be made with any suitable material. In one embodiment, the holding part can be made of a rigid material. In one embodiment, the holding part can be made of a flexible material with memory, for example PVC. In an embodiment, the U-shape can be formed by a one piece element. In the embodiment of the device depicted in FIG. 3, the holding part (2) optionally comprises a piece of deformable material (5). Deformable material (5) provides one or more gripping grooves (7) for receiving and holding various items such as tubes. In certain aspects, a rubber or foam material, or other material, that provides for expansion (to receive an item) and retention (e.g. frictional retention of the item) may be used. Such materials are advantageously typically naturally conforming to the shape of the item being held and provide enhanced tension. An example of such a deformable material with memory, includes a rubber or foam material that comprises the groove (7) to receive, hold and secure items such as tubes and cables/wires. Any type of deformable material with similar properties known in the art can be used.

Referring to FIG. 4, an embodiment of the device (1) is shown when in use, holding by way of illustration, electric cables and/or fluid tubes (6). FIG. 4 shows a portion (2) of a hospital bed, for example a handlebar or bed rail to which the clipping part attaches.

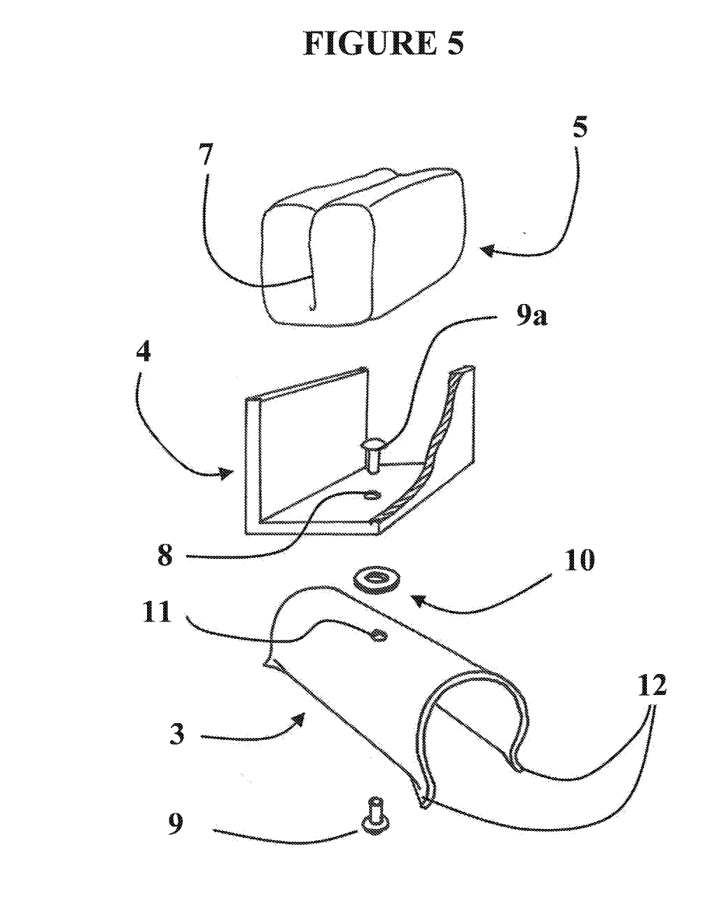

Referring to FIG. 5, an exploded view of an embodiment of the device (1) is shown. For a better view, a piece of the holding part (4) is shown with one portion cut away, so as to show an opening (8) in the bottom part of the holding part, through which connecting means, such as for example a rivet ((9a, 9b), male and female parts), connect the holding part (4) to the attachment element, e.g., clipping part (3) as shown. In embodiments where a mounting base element is used, the rivet connects the holding part to the mounting base element in a rotatable manner.

In one embodiment, a gasket element (10) is placed between the holding part (4) and the attachment element, e.g., clipping part (3), so as to constrain rotation in a way that rotation is controlled by hand and can resist forces exerted by the hospital items held by the holding part. The gasket can be a common rubber or non-skid constraining element or material. FIG. 5 also shows an opening (11) in the clipping part (3) to allow connection of the holding part and clipping parts by the connecting means, e.g. a rivet (9a, 9b). In an embodiment, the edge (12) of clipping part (3) has a rounded edge, and/or is slightly bent or comprises a slight bend, or is flared outward to allow easier clipping to a portion of a hospital bed (2).

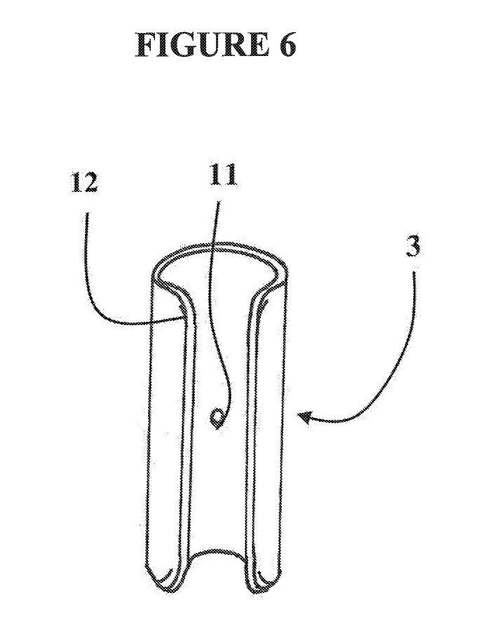

Referring to FIG. 6, a perspective view of another embodiment of clipping part (3) with opening (11) is shown. In this embodiment, the edges (12) of the clipping part are rounded to facilitate easier clipping to a portion of a hospital bed frame or other element.

Referring to FIG. 7, cross-sectional views of various embodiments of clipping part (3) are shown. These various embodiments are designed to be clipped on various commercially available hospital bed models and brands having elements such as bed rails, handlebars or other elements with corresponding or similar cross-sectional shapes. Drawings 7A to 7E show possible shapes of clipping parts according to various embodiments. Drawings 7A to 7D show shapes of clipping parts that can be suitable to hospital bed of the brand Stryker®. It is to be understood that the clipping part can have other shapes to accommodate other brands and other bed models, for example Hill-Rom® bed models. Also as shown in FIGS. 7B to 7F, in one embodiment, edge 12 has a rounded cross-sectional profile. FIG. 7A shows a clipping part with a similar cross-sectional profile to FIG. 7F, but with a non-rounded edge profile. It should be understood that each of the designs shown in FIG. 7 may have a non-rounded edge profile or a rounded edge profile. A rounded edge profile facilitates attachment to the portion of the bed frame. Also, each of the designs may include a rounded profile on one edge and a non-rounded profile on the other edge. Further, an edge may be flared out at an end of the cylinder (e.g., as shown in FIGS. 3 and 5 without a rounded profile) and also include a rounded profile.

Also, as shown in FIG. 7, certain designs include a locking cleat portion 20 to provide enhanced retention and stability (e.g., helps prevent rotation of clipping part on bed rail) to a portion of a bed frame such as a bed rail. It should be appreciated that a clipping part 3 may include a locking cleat portion 20 on both sides of the opening in the clipping part 3, e.g., as shown in FIGS. 7B to 7E, or on only one side of the opening in the clipping part 3. It should also be appreciated that the locking cleat may extend along the entire length of the edge of the cylinder, or may extend along only a portion of the edge. In certain embodiments, the clipping part may have a substantially custom fit (e.g., cross-sectional shape that matches that of the bed rail or other element to which the clipping part attaches).

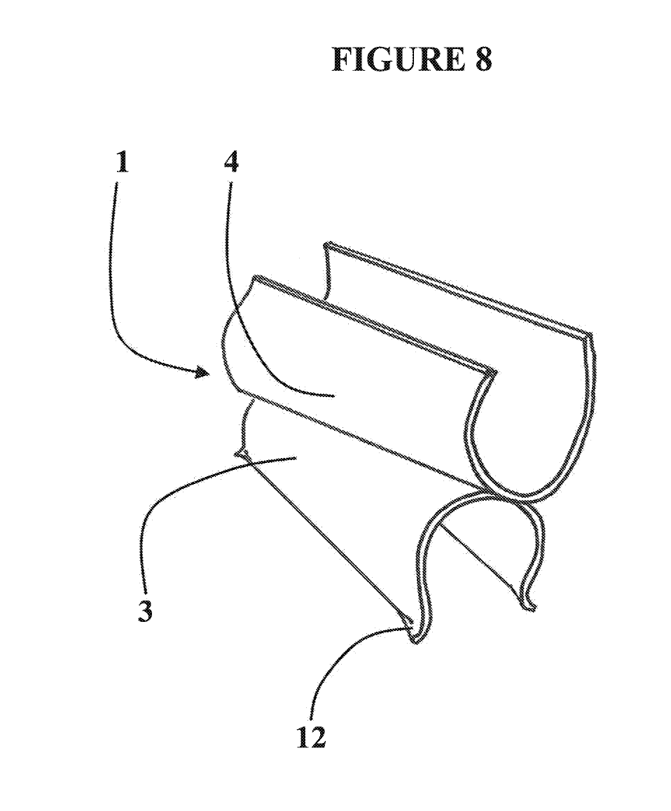

Referring to FIG. 8, a perspective view of an embodiment of a device (1) is shown. In this embodiment, the holding part (4b) is a cylinder that has a circular or nearly circular cross section in a plane that is perpendicular or nearly perpendicular to the length axis of said cylinder. This design allows the holding part to receive and hold a cylindrical or tubular shaped structure such as, for example, an oxygen tube, since most oxygen tubes have a circular or near circular cross-section. Although not shown, it should be appreciated that the edges of the holding part may be configured with similar profiles as the clipping part (e.g., rounded or non-rounded profiles, flared profiles, etc.) discussed above with respect to FIGS. 5-7.

In any of the embodiments herein, the holding part (4) can be made of any suitable material. In one embodiment the holding part can be made of a rigid material. In one embodiment the holding part can be made of a flexible material with memory, for example plastic material, such as PVC. If the holding part is made of flexible material with memory, an item such as for example an oxygen tube can conveniently be clipped into the holding part so as to be secured and held in place. In an embodiment, the holding part (4) can rotate with respect to the clipping part (3) and can be built in a similar way as other embodiments described herein, by replacing the substantially U-shaped holding part (3) of FIG. 3 with a cylindrical holding part (3) of FIG. 8. In this embodiment, a gasket can also be used to constrain rotation as described above.

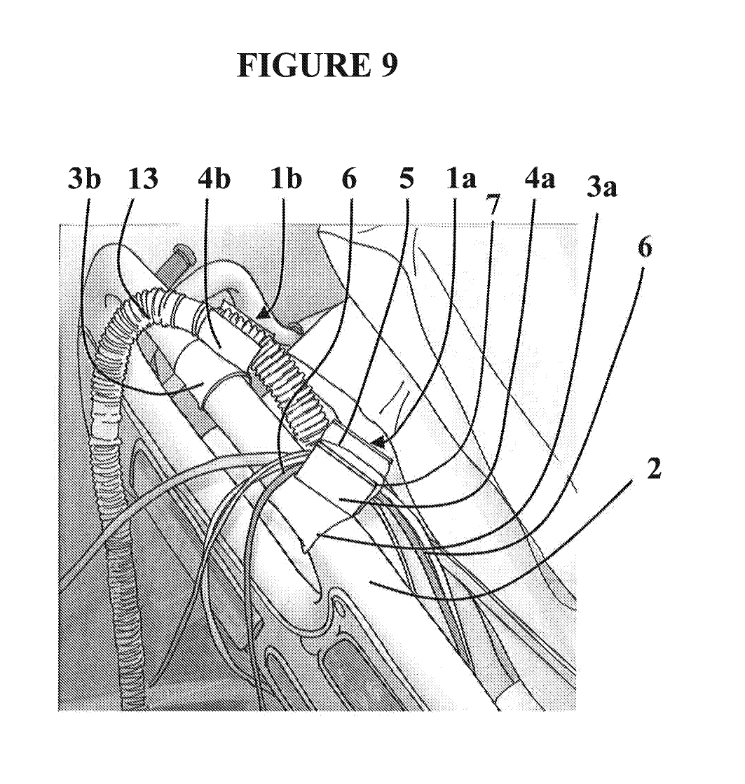

Referring to FIG. 9, a device (1) while in use clipped to a portion of a hospital bed is shown. The embodiment of FIG. 8 is shown with reference (1b). The embodiment of FIG. 3 is shown with reference (1a). Device (1b) comprises a holding part (4b) that holds an oxygen tube (13) and is clipped to a portion of a hospital bed (2) via its clipping part (3b). As shown on FIG. 9, the holding part (4b) is slightly rotated with respect to clipping part (3b) to accommodate proper placement of the oxygen tube (13) (e.g., proper orientation of the directional axis of the oxygen tube). Device (1a) comprises a holding part (4a) that comprises a piece of deformable material, e.g. a piece of foam (6) with a groove (7) holding and securing electric cables and fluid tubes (6) in place. Device (1a) is clipped to a portion of a hospital bed (2) via its clipping part (3a). As shown on FIG. 9, the holding part (4a) can be slightly rotated with respect to clipping part (3a) to accommodate proper placement and orientation of the cables and tubes (6). FIG. 9 shows how various embodiments of the device according to the invention can adequately be combined to properly hold in place hospital items in a safe manner for patients and hospital personnel.

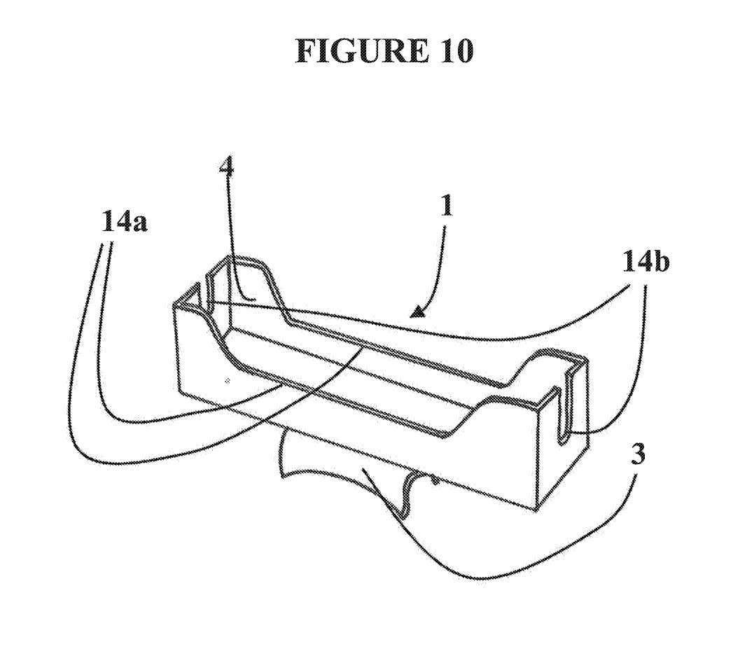

Referring to FIGS. 10, 11 and 12, yet another embodiment of a device (1) for holding hospital items, for example a bed or television remote control is shown.

Referring to FIG. 10, the holding part (4) has the shape of an open rectangular box wherein opposite side walls comprises recesses (14a) for easier access to the item held therein, for example a remote control. Additional recesses (14b) can be made in side walls to accommodate a wire or cable, such as for example a cable to connect the remote controller. In the embodiment shown on FIG. 10, the clipping part (3) is similar to the clipping part of other embodiments and can be made of the same material, but it is to be understood that the clipping part can be different and can for example have a cross section such as depicted on FIG. 7. Any embodiment that fulfills the clipping function of the clipping part described therein is also encompassed by the present disclosure.

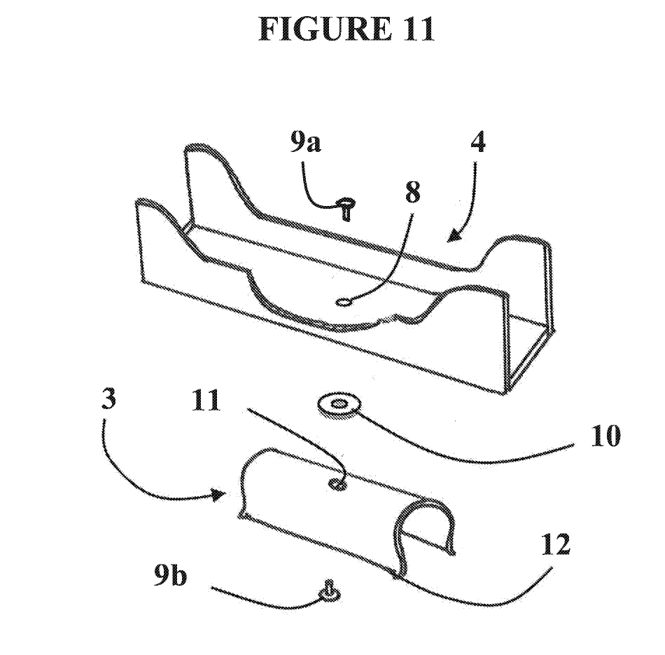

Referring to FIG. 11, for better illustration and explanation a portion of one of the side walls is shown cut away to show a possible location for an opening (8) through which a rivet (9a, 9b, male and female parts) connects the holding part (4) to the clipping part (3) via opening (11) on the clipping part (3). A gasket (10) can be added on the rivet between holding part (4) and clipping part (3) so as to constrain rotation, as described herein. In an embodiment, the edge (12) of clipping part (3) has a rounded edge, and/or is slightly bent or comprises a slight bend, so as to allow easier clipping to a portion of a hospital bed (2).

In any of the embodiments connection between the attachment element, e.g., clipping part (3), and the holding part (4) can be made in a manner so that attachment element does not rotate with regard to holding part (4). This can be achieved by any connecting means known in the art, such as using rivets, nuts and bolts, glue, welding, etc.

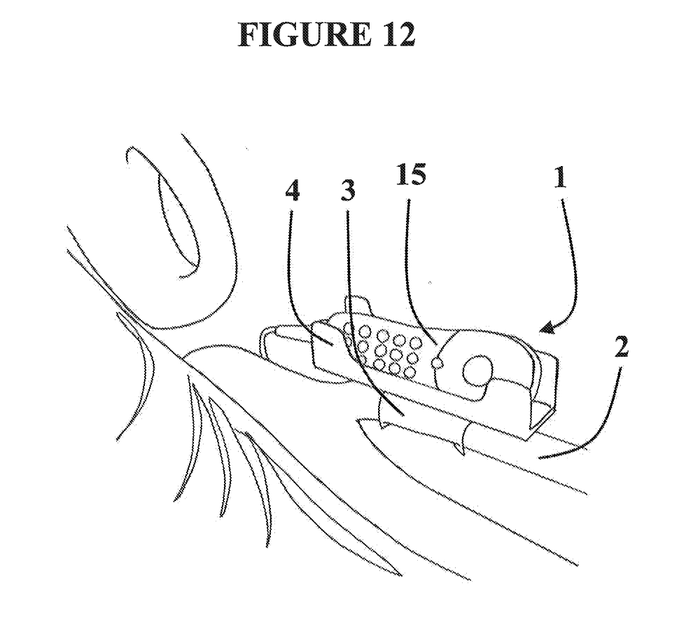

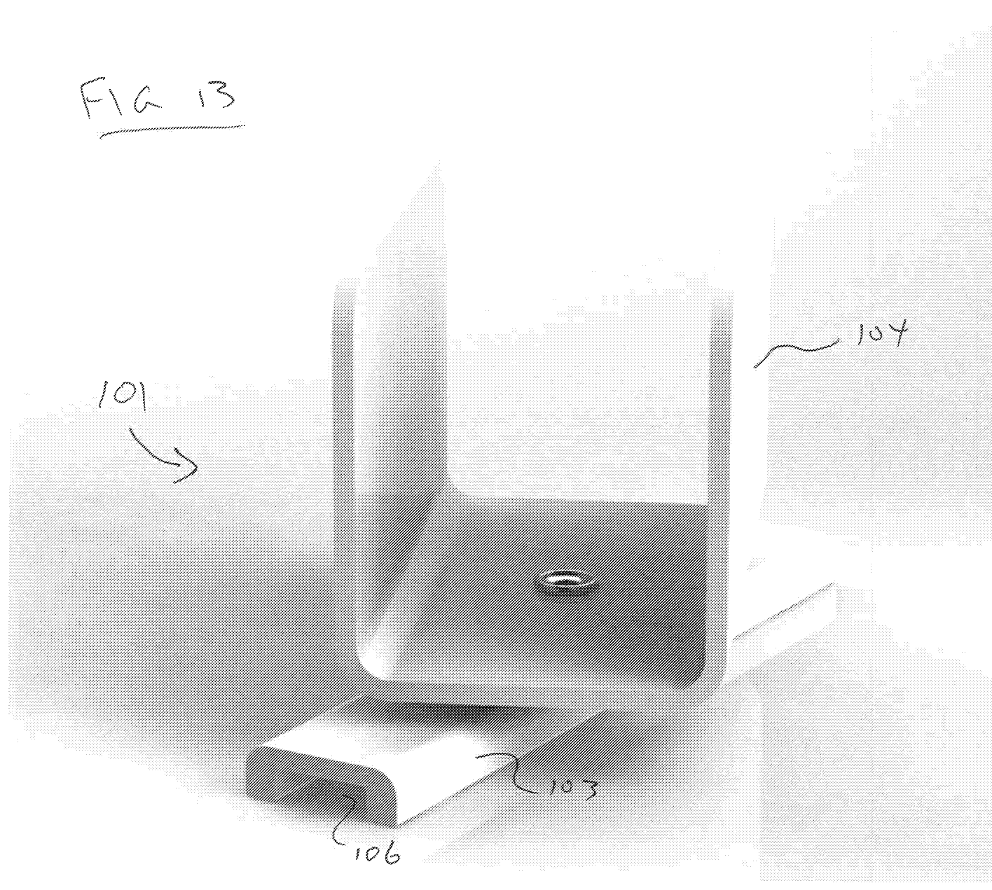

Referring to FIG. 12 an embodiment of a device is shown when in use, clipped to a portion (2) of a hospital bed frame. Holding part (4) can hold a bed remote control (15). As explained above, holding part (4) has side walls that comprise recesses (14a) (reference shown on FIG. 10) to allow easier access to the remote control unit held therein. Clipping part (3) is clipped to a portion (2) of a hospital bed frame.

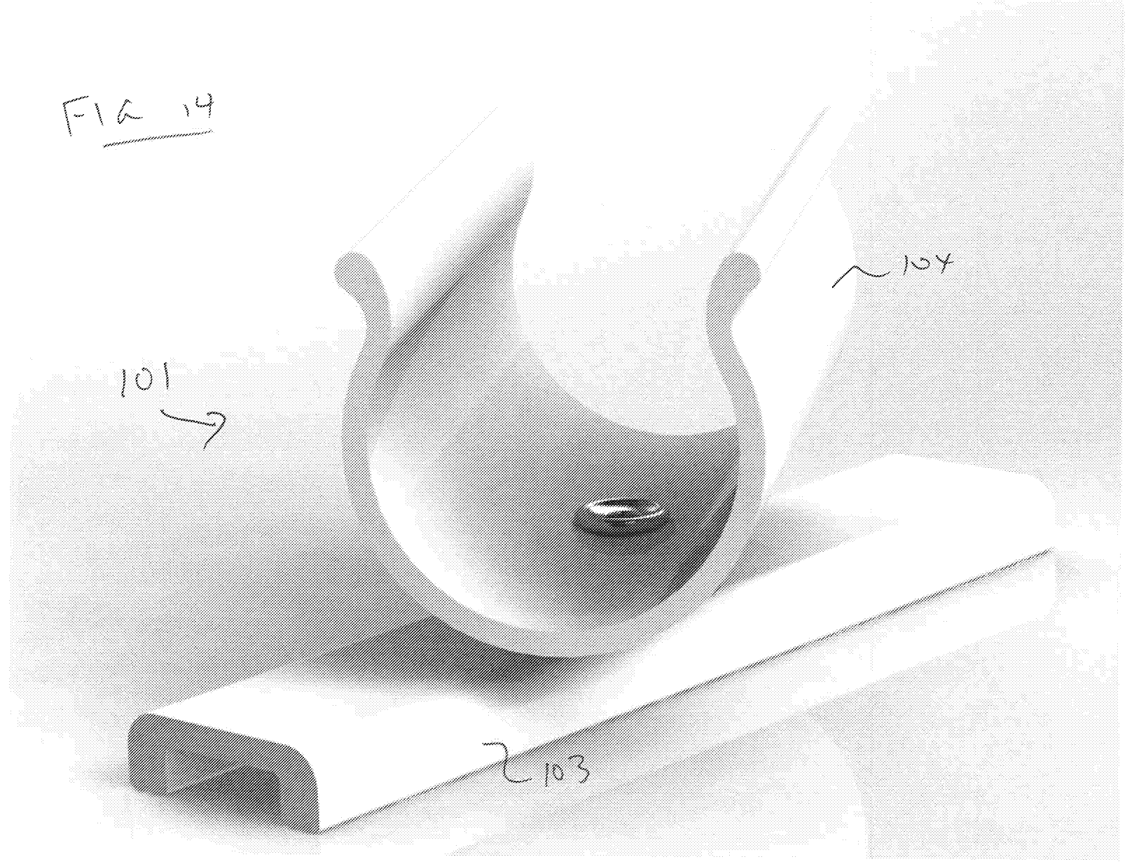

FIG. 13 illustrates a retention device 101 according to an embodiment. The device 101 includes an attachment element that comprises a mounting base portion 103 having a groove or channel 106 on an underside that contacts the portion of the bed frame. The mounting base can be made of a single piece of material, or multiple pieces attached or bonded together. The mounting base 103 can be made of rigid or semi-rigid material or a material that can be flexible with memory, such as polyvinyl chloride (PVC) or other plastic material that has similar physical properties. Extruded or molded PVC pieces can be used. In certain aspects, the clipping part (3) includes a portion (12) that is slightly bent toward the outside (or flared outward relative to its center length axis) at the edges of the opening so as to facilitate mating to a receiving element such as a bed rail that has a slightly curved, curved or rounded surface profile.

The device 101 further includes a holding part 104 connected to the mounting base 103 and designed for holding at least one item (not shown on FIG. 3). Connection of the mounting base 103 with the holding part 104 can be done in any of a variety of ways as is discussed above with reference to other embodiments. For example, in an embodiment, the holding part 104 is designed to be able to rotate with regard to the mounting base 103. This can, for example, be achieved by connecting the holding part 104 and the mounting base 103 with a connection element or connecting means that allow for a rotation of the holding part relative to the clipping part, for example and without limitation, a rivet, nut and bolt, etc. as is discussed above.

In the embodiment shown on FIG. 13, the holding part 104 is substantially U-shaped (cross-sectional shape) with two parallel side walls connected to a bottom wall (bottom refers to the direction of the device when in typical use). The holding part can be made with any suitable material. In one embodiment, the holding part can be made of a rigid material. In one embodiment, the holding part can be made of a flexible material with memory, for example PVC. In an embodiment, the U-shape can be formed by a one piece element. In the embodiment of the device depicted in FIG. 13, the holding part (2) optionally comprises a piece of deformable material (not shown, but see FIG. 3, element 5 as described above as an example). The deformable material provides one or more gripping grooves (e.g., element 7 of FIG. 3) for receiving and holding various items such as tubes. In certain aspects, a rubber or foam material, or other material, that provides for expansion (to receive an item) and retention (e.g. frictional retention of the item) may be used. Such materials are advantageously typically naturally conforming to the shape of the item being held and provide enhanced tension. An example of such a deformable material with memory, includes a rubber or foam material that comprises the groove (7) to receive, hold and secure items such as tubes and cables/wires. Any type of deformable material with similar properties known in the art can be used.

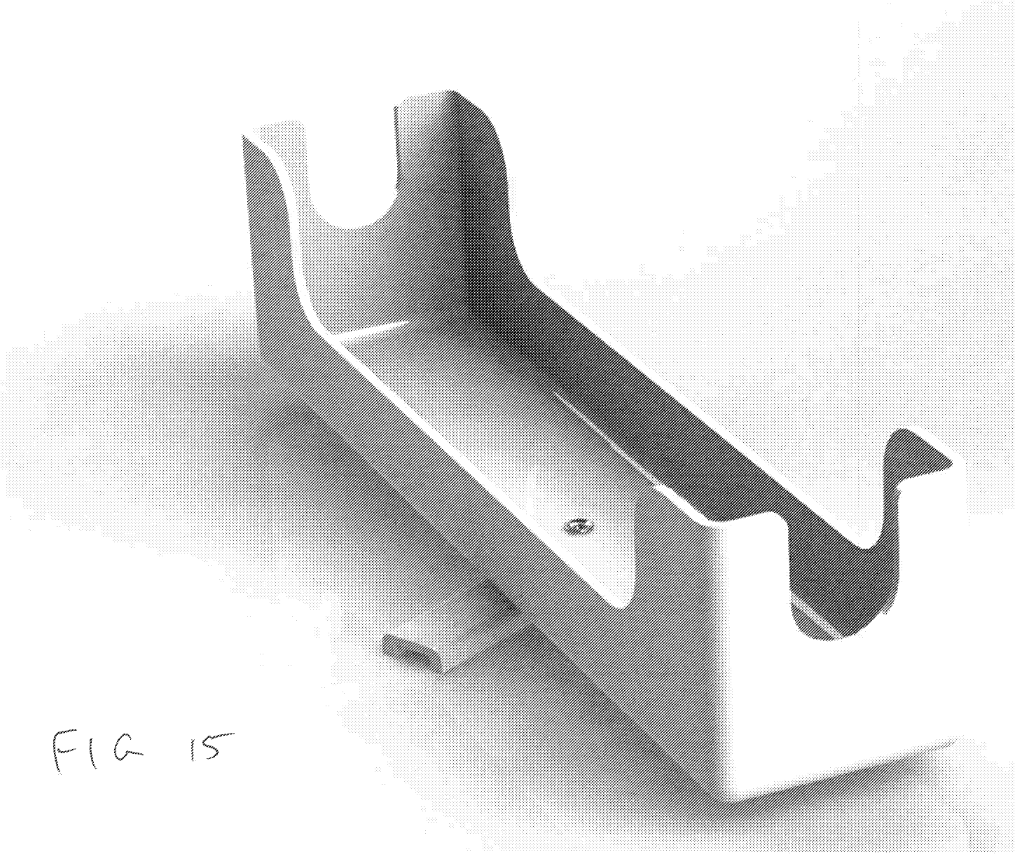

FIG. 14 shows an embodiment of a retention device 101 including a holding part 104 configured similar to the holding part 4 shown and described above with reference to FIG. 8.

FIG. 15 shows an embodiment of a retention device 101 including a holding part 104 configured similar to the holding part 4 shown and described above with reference to FIG. 10.

In certain embodiments, the attachment device 101 (e.g., as shown in FIGS. 13-15) may be coupled with or connected with a portion of a bedframe, such as a bed rail, using an adhesive mechanism such as a tape element. the tape element may include a strip of cohesive tape (e.g., a tape that is configured to stick or adhere to itself when overlapping itself such as when wrapped around an object, but which may have a reduced stickiness or lack a sticky/gummy surface material for sticking to other objects), a strip of Velcro, or a conventional tape material having a gum. Embodiments using a cohesive tape or velcro advantageously provide a robust attachment without leaving a gummy or sticky residue on the portion of the bedframe upon removal of the adhesive mechanism. Embodiments using a cohesive tape may provide better retention than Velcro strips as the cohesive tape allows for a slightly better retention to the bedframe portion without swiveling around the portion of the bedframe. The groove or channel 106 also advantageously allows for better retention on portions of a bedframe having a curved or rounded surface, e.g., allowing a better fit and reducing or preventing rotation on a curved surface.

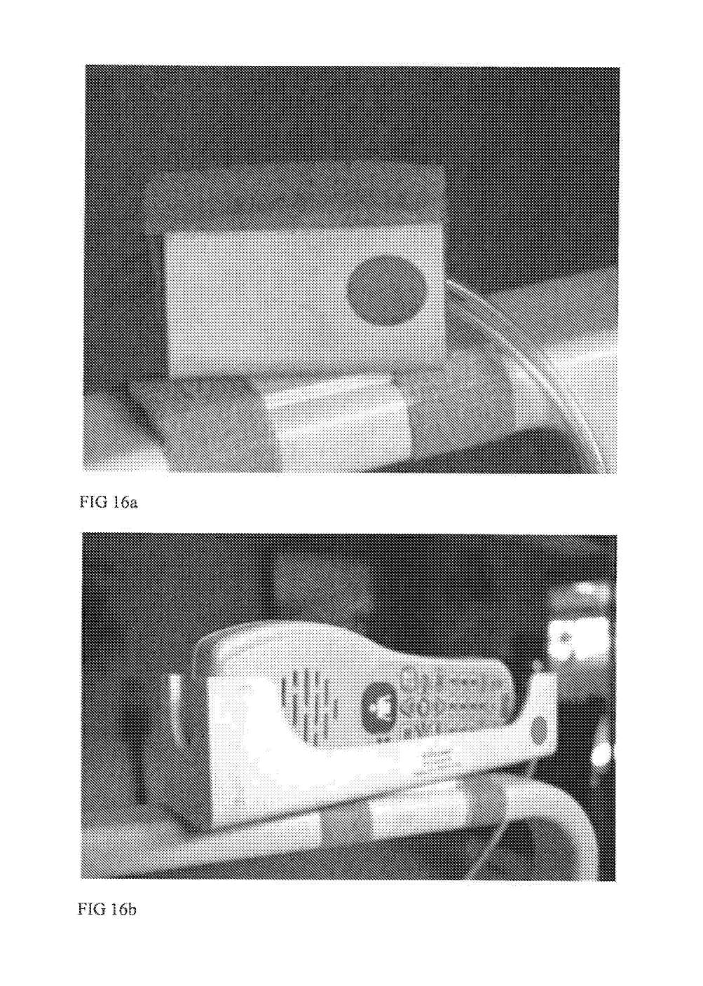

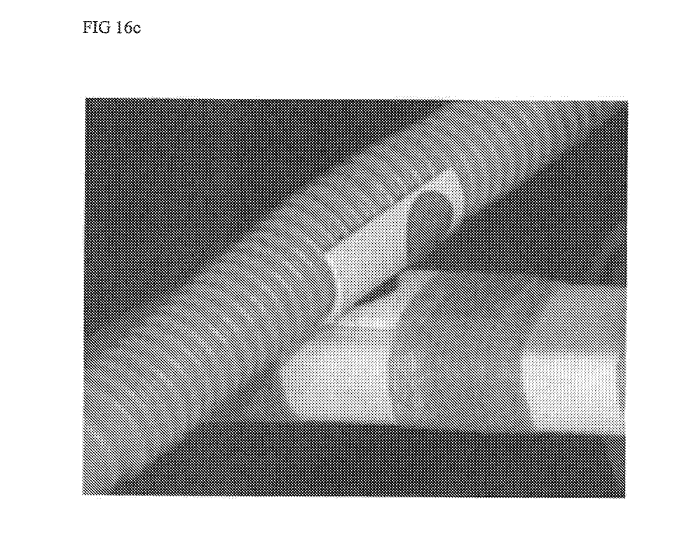

FIG. 16 illustrates embodiments of retention device 101 secured to a bedrail using a cohesive tape.

In one embodiment, a kit including components for making or forming a retention device (e.g., device 1 or device 101) is provided. In certain embodiments, the kit comprises an attachment element (e.g., element 3 or element 103) adapted to attach to a portion of a hospital bed frame, and a holding element (e.g., element 4 or element 104) adapted to be rotatably connected to the attachment element, wherein the holding element is able to rotate with regard to the attachment element when connected thereto. The holding element may include a receiving and retention element (e.g., a clipping structure such as element 104 or element 4, or an extra element 7) for holding at least one item. The kit also includes an adhesive mechanism. The adhesive mechanism may include a tape element adapted for securing the attachment element to the portion of the bed frame. The tape element may include a cohesive tape. The attachment element may include a substantially flat mounting base portion having a groove or channel on an underside adapted to contact the portion of the bed frame. The holding element may include a receiving and retention element comprised of a foam or rubber material to hold the at least one item. The receiving and retention element may include a groove for receiving and retaining the at least one item. The kit may further include a rivet or other attachment component adapted to connect the attachment element to the holding element in a rotatable manner, or the attachment element may be rotatable connected to the holding element (e.g., pre-built with the attachment component/rivet coupling the two pieces).

In one embodiment, the device (1) for holding items on a portion of a hospital bed (2) comprises:
a clipping part (3) comprising an elongated hollow and open cylinder made of a flexible material designed for clipping to said portion of a hospital bed (2);
a holding part (4) connected to said clipping part (3) and designed for holding remote control (5), said holding part (4) being permanently fixed to the clipping part (3).

In this embodiment, the device according to the present disclosure is especially suitable for holding a remote control as defined herein. In this embodiment, the clipping part does not rotate with regard to the holding part. In an embodiment, the clipping and the holding part are a one piece element. In an embodiment, the clipping part and the holding part are a one piece molded element.

It is to be understood that the devices according to the present disclosure can be secured to any suitable portion of a hospital bed, on the left or right side of the bed, relative to the patient.

### EXAMPLES

The following examples and figures are provided to aid the understanding of the present disclosure, the true scope of which is set forth in the appended claims. It is understood that modifications can be made in the procedures set forth without departing from the spirit of the invention.

### Example 1

### First Embodiment

A first embodiment of a device (1) according to the invention for holding hospital items, for examples electric cables or fluid tubes is described hereinafter with reference to FIGS. 3, 4, 5, 6, 7 and 9.

FIG. 3 shows an embodiment of a device (1) for holding items on a portion of a hospital bed (not shown on FIG. 3) comprising a clipping part (3) that comprises an elongated hollow and open cylinder made of a flexible material designed for clipping to said portion of a hospital bed. The device (1) further comprises a holding part (4) connected to the clipping part (3) and designed for holding at least one item (not shown on FIG. 3), said holding part (4) being able to rotate with regard to the clipping part (3). In this embodiment, the holding part (4) has a U-shape with two parallel side walls connected to a bottom wall. In an embodiment, the U-shape part can be a one piece element. In the embodiment depicted in FIG. 3, the holding part (2) optionally comprises a piece of deformable material (5), such as a deformable material with memory, for example a piece of foam, that comprises a groove (7) to receive, hold and secure items such as tubes.

FIG. 4 shows the same device (1) according to the invention shown in FIG. 3, when in use holding cables and/or tubes (6). FIG. 4 shows a portion (2) of a hospital bed, for example a handlebar.

FIG. 5 shows an exploded view of the same device (1) as shown in FIGS. 3 and 4. For a better view, a piece of the holding part (4) is cut away, so as to show an opening (8) in the bottom part of the holding part, through which connecting means, such as for example a rivet ((9a, 9b), male and female parts), connect the holding part (4) to the clipping part (3). In one embodiment, a gasket (10) is placed between the holding part (4) and the clipping part (3) so as to constrain rotation in a way that rotation is controlled by hand and can resist forces exerted by the hospital items held by the holding part. FIG. 5 also shows an opening (11) in the clipping part (3) to allow connection of the holding part and clipping parts by connecting means, e.g. a rivet (9a, 9b). In an embodiment, the edge (12) of clipping part (3) can be rounded, and/or slightly bent or comprise a slight bend, to allow easier clipping to a portion of a hospital bed (2).

FIG. 6 shows a perspective view of another embodiment of clipping part (3) with opening (11), wherein the edges of the clipping part are rounded for easier clipping to a portion of a hospital bed.

FIG. 7 shows a cross-sectional view of various embodiments of clipping part (3) adapted to be clipped on correspondingly shaped components of various hospital bed models. Drawings 7A to 7D on FIG. 7 show possible shapes of clipping parts that can be suitable for hospital beds of the brand Stryker®. It is to be understood that other the clipping can have other shapes to accommodate other brands and other bed models, for example Hill-Rom® models.

FIG. 9 shows two embodiments of devices (1) according to the invention while in use clipped to a portion of a hospital bed. A first embodiment of a device (1a) corresponds to the embodiment described herein in FIG. 3. In the embodiment according to FIG. 3, the holding part is shown as reference (4a), the clipping part as reference (3a). The second embodiment of a device (1b) according to the invention corresponds to the embodiment described herein in FIG. 8 herein below.

It is to be understood that other embodiments of the device are possible, even if not described specifically, within the scope of the present disclosure. Furthermore combinations of two or more embodiments of devices can be used simultaneously or separately on one or more hospital beds.

### Example 2

### Second Embodiment

A second embodiment of a device (1) for holding hospital items, for example an oxygen tube is described with reference to FIGS. 8 and 9.

FIG. 8 shows a perspective view of an embodiment of a device (1). In this embodiment, the holding part (4b) is a cylinder that has a cylindrical cross section so as to receive and hold an oxygen tube, since most oxygen tubes have a cylindrical cross-section. In this embodiment, the holding part (4) can rotate with respect to the clipping part (3) and can be built in a similar way as the embodiment described above, by replacing the U-shaped holding part (3) of example 1 by a cylindrical holding part (3). In this embodiment, a gasket can also be used to constrain rotation as described herein in example 1.

FIG. 9 shows two embodiments of devices (1) while in use, clipped to a portion (2) of a hospital bed. One embodiment is shown with reference (1b). Another embodiment is shown with reference (1a). Device (1b) comprises a holding part (4b) that holds an oxygen tube (13) and is clipped to a portion of a hospital bed (2) via its clipping part (3b). As shown on FIG. 9, the holding part (4b) is slightly rotated with respect to clipping part (3b) to accommodate proper placement of the oxygen tube (13). Device (1a) comprises a holding part (4a) that comprises a piece of deformable material, e.g. a piece of foam (6) with a groove (7) holding and securing electric cables and fluid tubes (6) in place. Device (1a) is clipped to a portion of a hospital bed (2) via its clipping part (3a). As shown on FIG. 9, the holding part (4a) is slightly rotated with respect to clipping part (3a) to accommodate proper placement of the cables and tubes (6) around the hospital bed. FIG. 9 shows how various embodiments of the device can adequately be combined to properly hold in place hospital items in a safe manner for patients and hospital personnel.

### Example 3

### Third Embodiment

A third embodiment of a device (1) for holding hospital items, for example a bed or television remote control is described hereinafter with reference to FIGS. 10, 11 and 12.

FIG. 10 shows a perspective view of a device (1). In this embodiment, the holding part (4) has the shape of an open rectangular box wherein the side walls comprise recesses (14a) for easier access to the item held therein by a remote control operator, for example for easier access for a patient to a remote control. Recesses (14b) allow passage for a cable when the remote control is wired through cables. This also allows for use on either side of a bed. In this embodiment, the clipping part (3) is similar to the clipping part of FIGS. 3 and 8, but it is to be understood that the clipping part can be different and can for example have a cross section such as depicted on FIG. 7.

FIG. 11 shows a perspective exploded view of the embodiment described in connection with FIG. 10. For better illustration and explanation a portion of one of the side wall of the holding is shown cut away to show a possible location for an opening (8) through which a rivet (9a, 9b, male and female parts) connects the holding part (4) to the clipping part (3) via opening (11) on the clipping part (3). A gasket (10) can be added on the rivet between holding part (4) and clipping part (3) so as to constrain rotation, as described herein. In an embodiment, the edge of clipping part (3) can be rounded, and/or slightly bent or comprise a slight bend (12), to allow easier clipping to a portion of a hospital bed (2).

FIG. 12 shows a perspective view of the embodiment described in connection with FIGS. 10 and 11 when in use, clipped to a portion of a hospital bed (2). Holding part (4) holds a bed remote control (15). As explained above, holding part (4) has side walls that comprise recesses (14) (reference shown on FIG. 10) for easier access to the remote control hold therein by a remote control operator, for instance the patient or a nurse. Clipping part (3) is clipped to a portion (2) of a hospital bed.

Although certain embodiments of the invention have been described using specific terms, such description is for illustrative purposes only, and it is to be understood that changes and variations may be made without departing from the spirit or scope of the following claims.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed:

1. A device for holding an item on a portion of a bed frame comprising:
an attachment element adapted to attach to said portion of a hospital bed frame; and
a holding element rotatably connected to said attachment element and including a receiving and retention element for holding at least one item, said holding element being able to rotate with regard to the attachment element.

2. The device of claim 1, wherein the attachment element comprises an elongated hollow and open cylinder made of a flexible or semi-flexible material that enables clipping to said portion of the bed frame.

3. The device of claim 1, wherein the attachment element includes a substantially flat mounting base portion having a groove or channel on an underside that contacts the portion of the bed frame.

4. The device of claim 3, further including a tape element that secures the attachment element to the portion of the bed frame.

5. The device of claim 4, wherein the tape element includes a cohesive tape.

6. The device of claim 1, wherein the holding element is an open rectangular box.

7. The device of claim 1, wherein the holding element has a substantially U-shaped cross section.

8. The device of claim 1 wherein the receiving and retention element includes a foam or rubber material.

9. The device of claim 8, wherein the receiving and retention element includes a groove for receiving and retaining the at least one item.

10. The device of claim 1, wherein the attachment element and the holding element are able to rotate with respect to one another in a plane that is perpendicular to an axis of the cylinder of the clipping part.

11. A device according to claim 1, wherein the at least one item is selected from the group consisting of a tube, an electrical cable, and a remote control unit.

12. A device according to claim 1, wherein the holding element comprises an elongated hollow and open cylinder made of a flexible or semi-flexible material that enables clipping to said at least one item.

13. The device of claim 1, further including a rivet that connects the attachment element to the holding element in a rotatable manner.

14. A kit including elements of a retention device for holding an item on a portion of a bed frame; the kit comprising:
an attachment element adapted to attach to a portion of a hospital bed frame;
a holding element adapted to be rotatably connected to said attachment element and including a receiving and retention element for holding at least one item, said holding element being able to rotate with regard to the attachment element when connected thereto; and
a tape element adapted for securing the attachment element to the portion of the bed frame.

15. The kit of claim 14, wherein the tape element includes a cohesive tape.

16. The kit of claim 14, wherein the attachment element includes a substantially flat mounting base portion having a groove or channel on an underside adapted to contact the portion of the bed frame.

17. The kit of claim 14, wherein the holding element includes a receiving and retention element comprised of a foam or rubber material to hold the at least one item.

18. The kit of claim 17, wherein the receiving and retention element includes a groove for receiving and retaining the at least one item.

19. The kit of claim 14, further including a rivet adapted to connect the attachment element to the holding element in a rotatable manner.

20. The kit of claim 14, wherein the attachment element is rotatable connected to the holding element.
